(19) United States
(12) Patent Application Publication
(10) Pub. No.: US 2026/0355313 A1
(43) Pub. Date: Nov. 12, 2026

(54) HYPOALLOGENIC-IMMUNOGENIC PLURIPOTENT STEM CELLS AS ANTI-CANCER VACCINE

(71) Applicant: The Board of Trustees of the Leland Stanford Junior University, Stanford, CA (US)

(72) Inventors: Lin Wang, Redwood City, CA (US); Joseph C. Wu, Stanford, CA (US)

(21) Appl. No.: 19/121,772

(22) PCT Filed: Nov. 14, 2023
(86) PCT No.: PCT/US2023/037236

§ 371 Date: Apr. 16, 2025

Related U.S. Application Data
(60) Provisional application No. 63/425,603, filed on Nov. 15, 2022.

(51) Int. Cl. A61K 40/10; A61K 35/545; A61K 40/42; A61P 35/00; C12N 15/86

(52) U.S. Cl. CPC A61K 40/10; A61K 35/545; A61K 40/4264; A61P 35/00; C12N 15/86; A61K 2239/39; A61K 2239/50; C12N 2740/16043

(57) ABSTRACT

Compositions and methods are provided for immunization against cancer cells, by the administration of non-self, hypoallogenic iPSCs. To reduce host rejections against the iPSCs after vaccination, the cells are engineered to knock out or otherwise reduce expression of MHC class I and MHC class II proteins. To further reduce rejection responses against the iPSCs after vaccination, the cells are engineered to over-express CD47. The tumor-associated antigens (TAAs) and tumor-specific antigens (TSAs) expressed by the engineered cells can activate both specific cellular immunity and humoral immune response to prevent tumor growth and/or eradicate tumor cells, in the absence of undesirable graft-versus-host diseases.

### CROSS REFERENCE TO OTHER APPLICATIONS

This application claims the benefit of U.S. Provisional Application No. 63/425,603, filed Nov. 15, 2022, the contents of which are hereby incorporated by reference in its entirety.

### GOVERNMENT RIGHTS

This invention was made with Government support under contracts 75N91019D00021 awarded by the National Institutes of Health. The Government has certain rights in the invention.

### BACKGROUND

Early embryonic gene products that are absent in adult organisms may be useful for the production of broad spectrum prophylactic cancer vaccines. Cancer cells and ESCs share many cellular and molecular features. These include a rapid proliferation rate, upregulated activity of telomerase, increased expression levels of oncogenes such as c-MYC and krupple-like factor 4 (KLF4), and similar overall gene expression profiles, microRNA signatures, and epigenetic status. Similar to cancer cells, after long-term culture the ESC lines will continue to proliferate actively and express high levels of telomerase activity, allowing them to maintain telomere length and cellular immortality. These features of ESCs resemble the hallmarks of cancer cells that have “sustaining proliferative signaling” and “replicative immortality”. There is abundant evidence that tumor cells and pluripotent stem cells can share antigens, and many types of neoplastic cells express certain embryonal antigens. Immune cells can be primed for anti-tumor responses by these embryonic antigens, which are not expressed in adult organisms and are not included in the T cell self-repertoire.

Syngeneic irradiated induced pluripotent stem cells (iPSCs) have been proposed in a prophylactic setting to vaccinate against “other” non-mutated neo-antigens, i.e., tumor antigens derived from proteins that are only expressed during embryonic development and not in adult tissues. For example, Kooreman et al. (Kooreman et al. Cell Stem Cell. 2018 April 5; 22 (4): 501-513.e7) immunized animals with irradiated syngeneic iPSCs in conjunction with the immunostimulatory CpG oligodeoxynucleotide as an adjuvant, obtaining a robust anti-tumor response in orthotopic mouse cancer models of breast cancer, melanoma and mesothelioma, and demonstrated that the vaccine confers long-term protection. Vaccination induced antibodies, CD4⁺ (T helper) and CD8⁺ cytotoxic T cell responses against tumor cells. iPSC vaccination in syngeneic mouse tumor models was well tolerated.

Improved cells for cancer vaccines are of great interest, and disclosed herein.

### SUMMARY

Compositions and methods are provided for immunization against cancer cells, by the administration of non-self (allogenic), hypoallogenic iPSCs. The hypoallogenic iPSCs express proteins that are expressed during embryonic development, which proteins can also be tumor-associated antigens (TAAs) and tumor-specific antigens (TSAs). To reduce host rejection against the allogenic iPSCs after vaccination, the cells are engineered to knock out or otherwise reduce expression of certain MHC class I and MHC class II proteins. To further reduce rejection response against the iPSCs after vaccination, the cells are engineered to over-express CD47. The tumor-associated antigens (TAAs) and tumor-specific antigens (TSAs) expressed by the engineered cells can activate both specific cellular immunity and humoral immune response to prevent tumor growth and/or eradicate tumor cells, in the absence of undesirable graft-versus-host diseases.

The hypoallogenic iPSCs are engineered to knock-out expression of Class I MHC proteins. In an embodiment, gene editing, e.g. CRISPR-Cas9 editing, is utilized to eliminate expression of $2 microglobulin, e.g. by deletion of all or a functional portion of the 32 microglobulin genes in the cell. The cells then lack detectable 32 microglobulin, which is required for cell surface expression of MHC class I proteins, e.g. HLA-A, HLA-B, HLA-C, etc. Levels of Class I MHC proteins may be non-detectable, or less than about 95%, less than about 98%, less than about 99% of the expression in the unmodified cell.

The hypoallogenic iPSCs are also engineered to knock-out expression of Class II MHC proteins. In an embodiment, gene editing, e.g. CRISPR-Cas9 editing, is utilized to eliminate expression of MHC II transactivator (CIITA) gene, e.g. by deletion of all or a functional portion of the CIITA genes in the cell. The cells then lack detectable CIITA, which regulates expression of Class II MHC proteins, e.g. HLA-DP, HLA-DQ and HLA-DR, etc. Levels of Class II MHC proteins may be non-detectable, or less than about 95%, less than about 98%, less than about 99% of the expression in the unmodified cell.

The hypoallogenic iPSCs are also engineered to over-express CD47. In an embodiment, an expression vector comprising a CD47 coding sequence operably joined to a promoter functional in the iPSC is introduced into the cells. In some embodiments the expression vector is a viral vector, e.g. a lentiviral vector. The cells then over-express CD47, where the level of CD47 on the surface is increased at least 1.5-fold, at least 2-fold, at least 3-fold, at least 5-fold, at least 10-fold or more higher than the expression level in the unmodified cell.

The hypoallogenic iPSCs may be selected prior to vaccine formulation for one or more of (i) decreased Class I MHC expression; (ii) decreased MHC Class Il expression; (iii) increased CD47 expression; and (iv) expression of one or more embryonic stem cell markers, e.g. SSEA-1 (CD15/Lewis x), SSEA-3, SSEA-4, D324 (E-Cadherin), CD90 (Thy-1), CD117 (c-KIT, SCFR), CD326, CD9 (MRP1, TM4SF DRAP-27, p24), CD29 (β1 integrin), CD24 (HAS), CD59 (Protectin), CD133, CD31 (PECAM-1), CD49f (Integrin α6/CD29), TRA-1-60, TRA-1-81, Frizzled5, Stem cell factor (SCF or c-Kit ligand), and Cripto (TDGF-1). In some embodiments the cells are selected for positive expression of an SSEA antigen, e.g. one or more of SSEA1, SSEA-3, SSEA-4.

The hypoallogenic iPSCs are generated from a somatic cell, for fibroblasts, nucleated blood cells, keratinocytes, etc., example see Rajasingh, J. Prog. Mol. Biol. Transl. Sci., 2012, 111:51-82 for a summary of the methods for the reprogramming of somatic cells and a method for regenerating patient-specific stem cells of any cell lineage without the use of embryonic stem cells. For example, sources of somatic cells for reprogramming may be selected from the group consisting of fibroblast, keratinocytes, peripheral blood cells and renal epithelial cells.

In one variation, the iPSCs are generated by genomic reprogramming using viral and nonintegrating nonviral methods. In some embodiments the hypoallogenic iPSCs are generated using a mini-intronic plasmid containing four reprogramming factors comprising Oct4, c-Myc, KLF-4 and Sox2, with the possible addition of shRNA p53. In an embodiment, where the pluripotent stem cells are genetically engineered to over-express one or more cancer antigens, e.g., CEA, MAGE-1, survivin, p53, HER2-neu, AFP, ras, etc. In some embodiments the hypoallogenic immunogenic iPSC are genetically engineered to downregulate one or more immune-suppressive proteins, e.g. TGF-beta, TFG-beta receptors. In other embodiments, hypoallogenic immunogenic iPSC are genetically engineered to overexpress pro-inflammatory proteins (eg. GM-CSF, IFN-gama, IFN-beta, etc.)

In an embodiment, methods for generation of a cancer vaccine are provided, where the vaccine targets multiple types of cancer, and are used by individuals other than the cell donor. A vaccine may comprise an adjuvant and hypoallogenic iPSCs as disclosed herein. In some embodiments the hypoallogenic iPSCs express cancer-related or cancer-associated epitopes that are common between iPSCs and cancer cells, and provide long-term immunity against the development and/or progression of cancer. Also provided is a vaccine for use in a method of treating cancer.

In an embodiment, the hypoallogenic iPSCs are combined with an adjuvant, where the adjuvant is an immunological agent to boost the immune response towards the vaccine. In one variation of the method, the adjuvant is selected from the group consisting of CpG, QS21, poly(di(carboxylatophenoxy)phosphazene; derivatives of lipopolysaccharides such as monophosphoryl lipid A, muramyl dipeptide (MDP; Ribi), threonyl-muramyl dipeptide (t-MDP; Ribi); OM-174; cholera toxin (CT), Leishmania elongation factor; or mixtures thereof. In some embodiments an adjuvant In other embodiments an adjuvant is selected from oil-in-water emulsion-based adjuvants (MF59, AS03), adjuvants containing the TLR4 agonist 3-O-desacyl-4′-monophosphoryl lipid A (MPL) (AS01, AS04), and CpG 1018, a TLR9 agonist CpG oligonucleotide.

In one embodiment, methods are provided for cancer vaccine generation and vaccination regimen, the methods comprising of in vitro generation of hypoallogenic iPSC cells and formulation with adjuvant, such as the pluripotent stem cells being combined with or emulsified in the adjuvant. In an embodiment, the vaccine is irradiated prior to vaccination. In an embodiment, the vaccination is performed weekly. In another embodiment, the vaccine is administered daily, several times a week such as twice or three times a week, or every two weeks, and the duration could be two, three, four, five, six, seven, or 8 weeks. In some embodiment the vaccine is administered weekly for at least 2 consecutive weeks, 3 consecutive weeks, 4 consecutive weeks, 5 consecutive weeks, at least 6 consecutive weeks or administered with additional boosters either one week, two week, three week, one month, two month or up to one year after the initial consecutive vaccination period. In some embodiments administration is less than or equal to 4 weeks.

In an embodiment, the vaccine is administered by subcutaneous injection. Alternatively, the vaccine is administered by intramuscular, intradermal, subcutaneous, intravenous, intraarterial, intrasplenic, intranodal, intratumoral or by intranasal methods.

In one embodiment, there is provided a method for the treatment of cancer in a patient, the method comprises a vaccination of the patient with a vaccine comprising an effective amount of hypoallogenic iPSCs as disclosed herein, wherein the vaccination comprises the step of administering hypoallogenic iPSCs to the patient in need thereof. As used herein, the method is equally applicable to any mammal that can be referred to as a patient for the treatment of cancer.

A therapeutically effective dose of the vaccine can boost or enhance the in vivo immune response by at least about 10%, at least about 20%, at least about 30%, at least about 50%, at least about 75%, at least about 90% or more, relative to the effect in the absence of administering the vaccine of the present application. The immune response may be at least one of a tumor specific response, an effective antigen presentation, a positive T-helper immune response and results in cytotoxic T-cell activities. Assays used to measure T-cell response include, but not limited to, delayed-type hypersensitivity testing, flow cytometry using peptide major histocompatibility complex tetramers, lymphoproliferation assay, enzyme-linked immunosorbant assay (ELISA), enzyme-linked immunospot assay (ELISpot), cytokine flow cytometry, cytotoxic T-lymphocyte (CTL) assay, CTL precursor frequency assay, T-cell proliferation assays, carboxyfluorescein diacetate succinimidyl ester assays, polyfunctional T-cell assays, measurement of cytokine mRNA by quantitative reverse transcriptase polymerase chain reaction (RT-PCR), and limiting dilution analysis. Other assays to evaluate immune responses include, but not limited to, gene expression profiling, protein microarrays to evaluate antibody responses to multiple antigens at one time, luciferase immunoprecipitation, phosphoflow for measuring multiple intracellular signaling molecules in the immune system at a single-cell level for lymphocyte immune monitoring, and surface plasmon resonance biosensors to monitor antibody immunity in serum.

In an embodiment, the vaccine is administered as a preventive therapy before cancer occurrence. In an embodiment, the vaccine is administered as an adjuvant therapy after tumor resection. In an embodiment, the vaccine is administered in conjunction with chemotherapy, other immunotherapy such as antibodies, and small molecules, including nanoparticles containing these agents or molecules. In an embodiment, the vaccine is given in the neo-adjuvant (before surgery), adjuvant (after surgery), or metastatic setting or before cancer develops in the preventative setting. In an embodiment of the method, the vaccine is administered as a neoadjuvant therapy before tumor resection. In another aspect of the method, the vaccine is administered as therapy in the metastatic setting. In another aspect of the method, the vaccine is administered in combination with single or multiple chemotherapeutic agents, immunotherapies, e.g. anti-PDL1, anti-PD1, or anti-CTLA4 antibodies, other biologics, and small molecules, e.g., diprovocim, including nanoparticles containing these agents.

Cancer types for treatment may include solid tumors, e.g., breast, lung, skin, glioblastoma, head & neck, thyroid, pancreatic, hepatic, colorectal, kidney, gastric, sarcoma, ovarian, bladder, prostate, esophageal, endometrial, cervical, as well as hematological cancers, e.g., Hodgkin's lymphoma, non-Hodgkin's lymphoma, multiple myeloma, myeloproliferative disorders, and leukemia. In an embodiments, the cancer is selected from the group consisting of breast cancer, melanoma and mesothelioma. In yet another aspect of the method, the cancer is selected from the group consisting of leukemia, multiple myeloma, lymphoma, myeloproliferative disorders, squamous cell cancer, adenocarcinoma, sarcoma, neuroendocrine carcinoma, bladder cancer, skin cancer, brain and spinal cord cancers, head and neck cancer, bone cancer, breast cancer, cervical cancer, colon cancer, rectal cancer, endometrial cancer, gastrointestinal cancers, (hypo) laryngeal cancer, esophageal cancer, germ cell cancer, transitional cell cancer, liver cancer, lung cancer, pancreatic cancer, cholangiocarcinoma, poorly differentiated carcinoma, prostate cancer, eye cancer, renal cell cancer, ovarian cancer, gastric cancer, testicular cancer, thyroid and thymus cancer.

In one variation of the method, the method of treatment results in no sign of autoimmune responses due to vaccine; or the method of treatment results in substantially no detectable sign of autoimmune responses due to vaccine. In another variation of the method, the vaccine is employed as an adjuvant therapy after tumor resection. In one variation, the method provides the patient with at least one adjuvant round; or at least two adjuvant rounds of (C+I) vaccine with no visible recurrence of the cancer, such as melanoma or breast cancer, or the cancers as recited herein. In another variation, the method results in an upregulation of mature antigen presenting cells (APCs) and an upregulation of helper T-cells.

In some embodiments, the vaccine reactivates the immune system in rejecting remnant cancer cells, such as breast cancer cells, by at least one of the systemic upregulation of IL-4 expressing B-cells, TNF-alpha expressing CD11b⁺GR1hi myeloid cells and a reduction of tumor-promoting Th17 cells. In another variation, the method results in inducing tumor degradation, including degradation near the tumor injection site and the vaccination site. In one variation, the method results in the reduction of the tumor size, by at least 10%, 20%, 30%, 40%, 50%, 75%, 85%, 90% or more than 95% after treatment. In another variation, the method results in the priming of the immune system and the reactivation of the immune system and specifically targets the cancer cells. In another variation of each of the above method, the method may be employed as adjuvant immunotherapy for multiple cancer types, and may be effective within 1 week, 2 weeks, 3 weeks, 4 weeks or within about 5 weeks after diagnosis. In another variation, the method provides a prophylactic immunization that results in an effective and specific response to multiple cancer types. In another variation, the effective and specific response results from an upregulation of mature APCs in the lymph nodes with a subsequent increase in helper T-cells and cytotoxic T-cells locally; and after a period of time, also an increase in helper T-cells and cytotoxic T-cells systematically. In another variation, B-cells and T-cells expressing IL-2, IL-4, and IL-5 may be predictive for tumor regression in the vaccination.

In another variation of the method, the vaccination creates broad tumor immunity against multiple cancer types and presents the immune system with large quantities (may include several dozens and up to hundreds or thousands) of tumor antigens. In another variation of the method, the vaccination reactivates the immune system in targeting established cancers without therapy-associated adverse effects, e.g., autoimmune response, weight loss, cytokine release syndrome and combination thereof.

In another embodiment, there is provided a thermally stable vaccine composition comprising an effective amount of hypoallogenic iPSCs as disclosed herein, and optionally, an adjuvant or an immunological agent to boost the immune response towards the vaccine. In one variation, the thermally stable vaccine or heat stable vaccine allows for storage that does not require cold chain storage, allowing the facile introduction of the vaccines in areas with no or limited cold chain storage capacity. In one variation, the vaccine further comprises an effective amount (such as an approximate range of 0.01% to 1% wt/wt, 0.05% to 0.5% wt/wt, 0.05% to 1% wt/wt, or 0.01% to 0.5% wt/wt) of a glycol, such as propylene glycol, polyethylene glycol 300 and glycerin, or mixtures thereof. In another variation, the vaccine is stable for up to 6 months, up to 12 months, up to 24 months or up to 36 months at about 35 °C, either as a standard liquid formulation or as a spray dried formulation. In an embodiment, the vaccine is formulated by lysate collected from 2 to 5 cycles of freezing and thawing lysis of hypoallogenic immunogenic iPSCs.

A benefit of the methods of the disclosure relative to existing cancer immunotherapies, such as checkpoint inhibitor-based immunotherapies or chimeric antigen receptor (CAR) T cells, hypoallergenic-immunogenic iPSCs based vaccine does not cause any immune related adverse events (irAEs) or organ-specific inflammatory side effects. In addition, it overcomes common drawbacks of individualized cell therapy, including manufacturing time, lack of Good Manufacturing Practice (GMP) conditions, and high cost. Thus, hypoallogenic immunogenic iPSCs provide an “off-the-shelf” stem cell therapy for a GMP standardized vaccine, which has the advantages of being cost-efficient and ready-to-use for cancer patients. Such an off-the-shelf vaccine can be produced in bulk as a universal vaccine and be used to treat a broad range of cancer patients.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. Hypoallogenic immunogenic iPSCs-based Vaccine Induces Anti-Colorectal Tumor Specific IgG Antibody Secretion in Mice After 4 weeks of Immunization Before Tumor Challenge.

FIG. 2. Mice that Received Hypoallogenic immunogenic iPSC-based Vaccine have Slower Tumor Growth After Syngeneic Colorectal Tumor Challenge.

FIG. 3. Hypoallogenic immunogenic iPSC-based Vaccine Inhibits Colorectal Tumor Growth.

FIG. 4. Splenocytes from CT26 Tumor-Bearing Mice that Pre-vaccinated with Hypoallogenic immunogenic iPSC-based Vaccine have Significant Increase in Proliferation Upon In-vitro CT26 Lysate Stimulation.

FIG. 5. Hypoallogenic immunogenic iPSC-based Vaccine Promoted Total IgG Secretion in Tumor Bearing Mice.

FIG. 6. No Vaccine-related Toxicities were Identified in Necropsy Analysis of Mice Immunized with Hypoallogenic immunogenic iPSC-based Vaccine.

FIGS. 7A-7B. Hypoallogenic immunogenic iPSC-based Vaccine Shows a Similar Anti-Cancer Efficacy as Autologous iPSC-based Vaccine.

FIG. 8. Similar to the Autologous-iPSC Vaccinated Group, Hypoallogeneic iPSC-vaccinated Group Produces Higher numbers of IFN-y Spots than the Other Vaccination Groups Under In-vitro Tumor Lysate Stimulation.

FIG. 9. Similar to the Autologous-iPSC Vaccinated Group, Hypoallogeneic iPSC-vaccinated Group Produces Higher numbers of Granzyme B Spots than Other Vaccination Groups Under In-vitro Tumor Lysate Stimulation.

FIG. 10. Hypoallogenic immunogenic iPSCs-based Vaccine Induces Anti-Colorectal Tumor Specific IgG Antibody Secretion in mice After 3 weeks of Tumor Challenge.

FIG. 11. Hypoallogenic immunogenic iPSCs-based Vaccine does not cause weight loss in mice.

FIG. 12. Hypoallogenic immunogenic iPSCs-based Vaccine does not Trigger Autoimmunity in mice.

FIG. 13. Hypoallo-immunogenic iPSC-based Vaccine Inhibits Primary Triple Negative Breast Tumor Growth in vivo.

FIG. 14. Hypoallo-immunogenic iPSC-based Vaccine Decreases Triple-Negative Breast Cancer Metastasis following Tumor Resection.

FIG. 15. Hypoallo-immunogenic iPSC-based Vaccine Decreases Triple-Negative Breast Cancer Metastasis following Tumor Resection.

FIG. 16. Hypoallo-immunogenic iPSC-based Vaccine Treatment reduces Growth of A20 tumor in Mice.

FIG. 17. Hypoallo-immunogenic C57BL6 iPSC line has B2m and Ciita gene knockout scores at 100 as well as the overexpression of CD47 protein compared to the control C57BL6 iPSC line.

FIG. 18A-18B. Hypoallo-immunogenic C57BL/6 iPSC line maintained high expression levels of pluripotency markers Sox2, Oct4, DPPA2

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

Compounds can also be made by methods known to one of ordinary skill in the art. As used herein, “methods known to one of ordinary skill in the art” may be identified through various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

“Comparable cell” shall mean a cell whose type is identical to that of another cell to which it is compared. Examples of comparable cells are cells from the same cell line.

“Inhibiting” the onset of a disorder shall mean either lessening the likelihood of the disorder's onset, or preventing the onset of the disorder entirely. In the preferred embodiment, inhibiting the onset of a disorder means preventing its onset entirely.

“Treating” a disorder shall mean slowing, stopping or reversing the disorder's progression. In the preferred embodiment, treating a disorder means reversing the disorder's progression, ideally to the point of eliminating the disorder itself. As used herein, ameliorating a disorder and treating a disorder are equivalent.

“Inhibiting” the expression of a gene in a cell shall mean either lessening the degree to which the gene is expressed or preventing such expression entirely. “Specifically inhibit” the expression of a protein shall mean to inhibit that protein's expression (a) more than the expression of any other protein, or (b) more than the expression of all but 10 or fewer other proteins.

The terms “subject,” “individual,” and “patient” are used interchangeably herein to refer to a mammal being assessed for treatment and/or being treated. In an embodiment, the mammal is a human. The terms “subject,” “individual,” and “patient” thus encompass individuals having cancer. Subjects may be human, but also include other mammals, particularly those mammals useful as laboratory models for human disease, e.g. mouse, rat, etc.

As used herein, the terms “treatment,” “treating,” and the like, refer to administering an agent, or carrying out a procedure for the purposes of obtaining an effect. The effect may be prophylactic in terms of completely or partially preventing a disease or symptom thereof and/or may be therapeutic in terms of effecting a partial or complete cure for a disease and/or symptoms of the disease.

Treating may refer to any indicia of success in the treatment or amelioration or prevention of cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician.

“In combination with”, “combination therapy” and “combination products” refer, in certain embodiments, to the concurrent administration to a patient of a first therapeutic (i.e., first therapeutic agent) and the compounds as used herein. When administered in combination, each component can be administered at the same time or sequentially in any order at different points in time. Thus, each component can be administered separately but sufficiently closely in time so as to provide the desired therapeutic effect. First therapeutic agents contemplated for use with the methods of the present invention include any other agent for use in the treatment of cancer.

“Concomitant administration” of a known therapeutic agent with a pharmaceutical composition of the present invention means administration of the therapeutic agent and inhibitor agent at such time that both the known therapeutic agent and the composition of the present invention will have a therapeutic effect. Such concomitant administration may involve concurrent (i.e. at the same time), prior, or subsequent administration of the drug with respect to the administration of a compound of the present invention. A person of ordinary skill in the art would have no difficulty determining the appropriate timing, sequence and dosages of administration for particular drugs and compositions of the present invention. Therapeutic agents contemplated for concomitant administration according to the methods of the present invention include any other agent for use in the treatment of cancer.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient ”means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

The phrase “determining the treatment efficacy” and variants thereof can include any methods for determining that a treatment is providing a benefit to a subject. The term “treatment efficacy” and variants thereof are generally indicated by alleviation of one or more signs or symptoms associated with the disease and can be readily determined by one skilled in the art. “Treatment efficacy” may also refer to the prevention or amelioration of signs and symptoms of toxicities typically associated with standard or non-standard treatments of a disease. Determination of treatment efficacy is usually indication and disease specific and can include any methods known or available in the art for determining that a treatment is providing a beneficial effect to a patient. For example, evidence of treatment efficacy can include but is not limited to remission of the disease or indication. Further, treatment efficacy can also include general improvements in the overall health of the subject, such as but not limited to enhancement of patient life quality, increase in predicted subject survival rate, decrease in depression or decrease in rate of recurrence of the indication (increase in remission time). (See, e.g., Physicians' Desk Reference (2010).)

“Major histocompatibility complex antigens” (“MHC”, also called “human leukocyte antigens”, HLA) are protein molecules expressed on the surface of cells that confer a unique antigenic identity to these cells. MHC/HLA antigens are target molecules that are recognized by T-cells and natural killer (NK) cells as being derived from the same source of hematopoietic stem cells as the immune effector cells (“self”) or as being derived from another source of hematopoietic reconstituting cells (“non-self”). Two main classes of HLA antigens are recognized: HLA class I and HLA class II. HLA class I antigens (A, B, and C in humans) render each cell recognizable as “self,” whereas HLA class Il antigens (DR, DP, and DQ in humans) are involved in reactions between lymphocytes and antigen presenting cells. Both have been implicated in the rejection of transplanted organs.

The HLA genes are clustered in a “super-locus” present on chromosome position 6p21, which encodes the six classical transplantation HLA genes and at least 132 protein coding genes that have important roles in the regulation of the immune system as well as some other fundamental molecular and cellular processes. The complete locus measures roughly 3.6 Mb, with at least 224 gene loci. One effect of this clustering is that “haplotypes”, i.e. the set of alleles present on a single chromosome, which is inherited from one parent, tend to be inherited as a group. The set of alleles inherited from each parent forms a haplotype, in which some alleles tend to be associated together. Identifying a patient's haplotypes can help predict the probability of finding matching donors and assist in developing a search strategy, because some alleles and haplotypes are more common than others and they are distributed at different frequencies in different racial and ethnic groups.

As used herein, the term “HLA matched” refers to a donor recipient pair in which none of the HLA antigens are mismatched between the donor and recipient. HLA matched (i.e., where all of the 6 alleles are matched) donor/recipient pairs have a decreased risk of graft v. host disease (GVHD) relative to mismatched pairs (i.e. where at least one of the 6 alleles is mismatched). HLA haploidentical refers to a match where one chromosome is matched at least at HLA-A; HLA-B and HLA-DR, and may be matched at minor histocompatibility loci on the chromosome; but is not necessarily matched on the second chromosome. Such donors frequently occur in families, e.g. a parent is haploidentical to a child; and siblings may be haploidentical.

The hypoallogenic iPSCs of the disclosure may be MHC mismatched relative to the vaccination recipient. As used herein, the term “HLA mismatched” refers to a donor recipient pair in which at least one HLA antigen, in particular with respect to HLA-A, HLA-B and HLA-DR, is mismatched between the donor and recipient. In some cases, one haplotype is matched and the other is mismatched.

HLA alleles are typically noted with a variety of levels of detail. Most designations begin with HLA-and the locus name, then * and some (even) number of digits specifying the allele. The first two digits specify a group of alleles. Older typing methodologies often could not completely distinguish alleles and so stopped at this level. The third through fourth digits specify a synonymous allele. Digits five through six denote any synonymous mutations within the coding frame of the gene. The seventh and eighth digits distinguish mutations outside the coding region. Letters such as L, N, Q, or S may follow an allele's designation to specify an expression level or other non-genomic data known about it. Thus, a completely described allele may be up to 9 digits long, not including the HLA-prefix and locus notation.

As used herein, a “recipient” is an individual to whom the allogenic iPSCs are administered. For the purposes of the present disclosure, a recipient and a donor may be HLA-matched or HLA-mismatched.

β2 microglobulin (B2M) is a component of MHC class I molecules. β2 microglobulin lies beside the α3 chain on the cell surface. Unlike α3, β32 has no transmembrane region. Directly above β2 (that is, further away from the cell) lies the α1 chain, which itself is next to the α2. β2 microglobulin associates not only with the alpha chain of MHC class I molecules, but also with class I-like molecules such as CD1 (5 genes in humans), MR1, the neonatal Fc receptor (FcRn), and Qa-1 (a form of alloantigen). Nevertheless, the β2 microglobulin gene is outside of the MHC (HLA) locus, on a different chromosome. β2 microglobulin is necessary for cell surface expression of MHC class I and stability of the peptide-binding groove. In the absence of β2 microglobulin, very limited amounts of MHC class I (classical and non-classical) molecules can be detected on the surface.

MHC-Class Il genes show a highly controlled developmental, cell-type and stimulus-specific expression with constitutive expression confined to professional antigen-presenting cells (APCs) such as dendritic cells (DCs), B cells, macrophages, and thymic epithelial cells. Other cell types are negative; however, the expression can be induced in diverse cell types by different stimuli, most prominently IFNγ. The class II transactivator (CIITA) has been recognized as the “master regulator” of MHC-II expression since the differential expression of MHC-II genes is largely due to the differential expression of CIITA. CIITA is a founding member of the NLR (nucleotide-binding and leucine-rich-repeat-containing) protein family, but for a long time, it stood apart in this protein family as the only transcriptional regulator. CIITA controls IFNγ-induced MHC-Il expression. CIITA controls MHC-II expression quantitatively, and there is a close correlation between levels of CIITA and MHC-II mRNA expression levels in different tissues.

The CIITA protein structure is characterized by an N-terminal acidic domain, a region rich in prolines, serines, and threonines (P/S/T domain), a central nucleotide-binding domain (GTP domain) and at least four C-terminal leucine-rich repeats (LRRs). Three alternative promoters (pl, pill, and plV) and corresponding exons one generate three different isoforms of CIITA (isoforms I, III, and IV) differing in their N-termini. Isoform IV is initiated by an AUG in the common exon two, whereas isoforms I and III carry their own initiator AUGs leading to N-terminal extensions of 101 and 24 amino acids, respectively. The N-terminal extension of isoform I shows homology to caspase activation and recruitment domain (CARD) and was found to increase MHC-II transcription.

CD47 is a 50 kDa transmembrane receptor that has extracellular N-terminal IgV domain, five transmembrane domains, and a short C-terminal intracellular tail. There are four alternatively spliced isoforms of CD47 that differ only in the length of their cytoplasmic tail. It binds to signal-regulatory protein alpha (SIRPα). The CD47/SIRPα interaction leads to bidirectional signaling, resulting in different cell-to-cell responses including inhibition of phagocytosis, stimulation of cell-cell fusion, and T-cell activation, and leads to its activity as a don't eat me signal to phagocytic cells of the immune system. For example, red blood cells that lack CD47 are rapidly cleared from the bloodstream by macrophages, a process that is mediated by interaction with SIRPα.

Sequences of human and mouse CD47 are publicly available, for example the human protein reference sequence at Genbank is NP_034711.1 and the mouse reference protein sequence is NP_001768.1.

For physiologically relevant purposes the binding of SIRPα and CD47 is usually an event between SIRPα on phagocytic cells and their precursors (e.g., macrophages and monocytes); and CD47 on articles, particularly particulate articles such as nanoparticles, microparticles, etc. that can be targets for phagocytosis.

Oncofetal antigens are proteins which are typically present only during fetal development but are found in adults with certain kinds of cancer. These proteins are often measurable in the blood of individuals with cancer and may be used to immunize, diagnose and follow treatment of the tumors.

α-fetoprotein (AFP) is associated with hepatocellular carcinoma, yolk sac tumor, colon and ovarian cancer. AFP-targeting immunotherapy could induce tumor-specific CTL. Human chorionic gonadotropin (HCG) is associated with colon, ovarian, lung and prostate cancer. Glypican 3 is associated with hepatocellular carcinoma and melanoma. Glypican-3 peptide vaccines induce specific CTLs in most patients. Cancer/testis antigen (CTA) is associated with melanoma, ovarian, lung, prostate, sarcoma and hepatocellular cancer and can find use as a cancer vaccine. Carcinoembryonic antigen (CEA) is associated with lung, colon, ovarian and breast cancer, and induces T cell responses. Immature laminin receptor (OFA-iLR) is associated with breast, lung, ovarian and prostate carcinoma, lymphoma and renal cell carcinoma. Placental alkaline phosphatase (PLAP) is associated with colon, ovarian, breast, lung, testicular and gastric cancer. CA125 is associated with ovarian cancer. IMP family (IGF2 mRNA-binding protein) is associated with non-small cell lung cancers and breast cancer.

Other embryonic stem cell surface markers, include, for example, SSEA-1 (CD15/Lewis x), SSEA-3, SSEA-4, D324 (E-Cadherin), CD90 (Thy-1), CD117 (c-KIT, SCFR), CD326, CD9 (MRP1, TM4SF DRAP-27, p24), CD29 (β1 integrin), CD24 (HAS), CD59 (Protectin), CD133, CD31 (PECAM-1), CD49f (Integrin α6/CD29), TRA-1-60, TRA-1-81, Frizzled5, Stem cell factor (SCF or c-Kit ligand), and Cripto (TDGF-1).

A tumor-associated antigen (TAA) or tumor-specific antigen (TSA), as used herein, refers to known and also unknown antigens/epitopes present on cancer cells. An optimal immune response with the cancer vaccine is to prime the host's immune system to target these TAAs and TSAs, present on pluripotent cells, and provide immunity to cancer types that express the TAAs and TSAs. Known TAAs and TSAs include, but are not limited to, EPCAM, CEACAM, TERT, WNK2, survivin, etc.

An adjuvant is an immunological agent that boosts the immunological response of the recipients' immune system to target the pluripotent stem cells. The adjuvant includes those disclosed in the present application and those known in the art for boosting the immunological response of the recipients' immune system to target the pluripotent stem cells. The term “adjuvant” refers to any substance or agent that can stimulate an immune response. Some adjuvants can cause activation of a cell of the immune system. For example, an adjuvant can cause an immune cell to produce and secrete a cytokine. Examples of adjuvants that can cause activation of a cell of the immune system include, but are not limited to, the nanoemulsion formulations described herein, saponins purified from the bark of the Q. Saponaria tree, such as QS21, poly(di(carboxylatophenoxy)phosphazene (PCPP polymer; Virus Research Institute, USA); derivatives of lipopolysaccharides such as monophosphoryl lipid A (MPL; RibilmmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); cholera toxin (CT), and Leishmania elongation factor (a purified Leishmania protein; Corixa Corporation, Seattle, Wash.); or a mixture thereof. Other adjuvants known in the art may include, for example, aluminum phosphate or hydroxide salts. In some embodiments, for example, the pluripotent stem cells of the present invention are administered with one or more adjuvants. In some embodiments, the adjuvants employed are described in US2005158329; US 2009010964; US 2004047882; or U.S. Pat. No. 6,262,029.

In some embodiments an adjuvant composition is selected for use in the vaccine. Exemplary adjuvants are oil in water emulsions, and may comprise squalene in the oil phase. For example, AS03 is an adjuvant system composed of α-tocopherol, squalene and polysorbate 80 in an oil-in-water emulsion. MF59 is another immunologic adjuvant that comprises a squalene emulsion. Usually the quantity is within the range conventionally used for adjuvants. For example, adjuvants typically comprises from about 1 mg to about 1000 mg, inclusive, of a 1-mL dose.

The adjuvant formulations can be homogenized or microfluidized. The formulations are subjected to a primary blending process, typically by passage one or more times through one or more homogenizers. Any commercially available homogenizer can be used for this purpose, e.g., Ross emulsifier (Hauppauge, N.Y.), Gaulin homogenizer (Everett, Mass.), or Microfluidics (Newton, Mass.). In one embodiment, the formulations are homogenized for three minutes at 10,000 rpm. Microfluidization can be achieved by use of a commercial mirofluidizer, such as model number 110Y available from Microfluidics, (Newton, Mass.); Gaulin Model 30CD (Gaulin, Inc., Everett, Mass.); and Rainnie Minilab Type 8.30H (Miro Atomizer Food and Dairy, Inc., Hudson, Wis.). These microfluidizers operate by forcing fluids through small apertures under high pressure, such that two fluid streams interact at high velocities in an interaction chamber to form compositions with droplets of a submicron size. In one embodiment, the formulations are microfluidized by being passed through a 200 micron limiting dimension chamber at 10,000+/−500 psi.

The routes of administration for the adjuvant compositions include parenteral, oral, oronasal, intranasal, intratracheal, topical, etc. Any suitable device may be used to administer the compositions, including syringes, droppers, needleless injection devices, patches, and the like. The route and device selected for use will depend on the composition of the adjuvant, the antigen, and the subject, and such are well known to the skilled artisan.

The adjuvant compositions can further include one or more immunomodulatory agents such as, e.g., quaternary ammonium compounds (e.g., DDA), and interleukins, interferons, or other cytokines. These materials can be purchased commercially. The amount of an immunomodulator suitable for use in the adjuvant compositions depends upon the nature of the immunomodulator used and the subject. However, they are generally used in an amount of about 1 μg to about 5,000 μg per dose. For a specific example, adjuvant compositions containing DDA can be prepared by simply mixing an antigen solution with a freshly prepared solution of DDA.

The adjuvant compositions can further include one or more polymers such as, for example, DEAE Dextran, polyethylene glycol, and polyacrylic acid and polymethacrylic acid (eg, CARBOPOL. RTM.). Such material can be purchased commercially. The amount of polymers suitable for use in the adjuvant compositions depends upon the nature of the polymers used. However, they are generally used in an amount of about 0.0001% volume to volume (v/v) to about 75% v/v. In other embodiments, they are used in an amount of about 0.001% v/v to about 50% v/v, of about 0.005% v/v to about 25% v/v, of about 0.01% v/v to about 10% v/v, of about 0.05% v/v to about 2% v/v, and of about 0.1% v/v to about 0.75% v/v. In another embodiment, they are used in an amount of about 0.02 v/v to about 0.4% v/v. DEAE-dextran can have a molecular size in the range of 50,000 Da to 5,000,000 Da, or it can be in the range of 500,000 Da to 2,000,000 Da. Such material may be purchased commercially or prepared from dextran.

The adjuvant compositions can further include one or more Th2 stimulants such as, for example, Bay R1005™ and aluminum. The amount of Th2 stimulants suitable for use in the adjuvant compositions depends upon the nature of the Th2 stimulant used. However, they are generally used in an amount of about 0.01 mg to about 10 mg per dose. In other embodiments, they are used in an amount of about 0.05 mg to about 7.5 mg per dose, of about 0.1 mg to about 5 mg per dose, of about 0.5 mg to about 2.5 mg per dose, and of 1 mg to about 2 mg per dose. A specific example is Bay R1005™, a glycolipid with the chemical name “N-(2-deoxy-2-L-leucylamino-β-D-glucopyranosyl)-N-octadecyldodecanamide acetate.” It is an amphiphilic molecule which forms micelles in aqueous solution. Other examples of useful adjuvant include, but are not limited to, complete and incomplete Freund's, mineral gels such as aluminum hydroxide, surface active substances such as lysolecithin, pluronic polyols, polyanions, peptides and oil emulsions.

As used herein, the clause “an amount effective to boost (or induce) an immune response” (for example, a composition for inducing or boosting an immune response), refers to the dosage level or amount required (for example, when administered to a mammal) to stimulate, generate and/or elicit an immune response in the mammal. An effective amount can be administered in one or more administrations over different time periods, as disclosed herein (for example, via the same or different route). The application or dosage is not intended to be limited to a particular formulation or an administration route or time period.

By “pluripotency” and pluripotent stem cells, it is meant that such cells have the ability to differentiate into all types of cells in an adult organism. The term “induced pluripotent stem cell” encompasses pluripotent cells, that, like embryonic stem cells (ESCs), can be cultured over a long period of time while maintaining the ability to differentiate into all types of cells in an organism, but that, unlike ESCs (which are derived from the inner cell mass of blastocysts), are derived from differentiated somatic cells, that is, cells that had a narrower, more defined potential and that in the absence of experimental manipulation could not give rise to all types of cells in the organism. By “having the potential to become iPSCs” it is meant that the differentiated somatic cells can be induced to become, i.e. can be reprogrammed to become, iPSCs. In other words, the somatic cell can be induced to redifferentiate so as to establish cells having the morphological characteristics, growth ability and pluripotency of pluripotent cells. iPSCs have an human ESC-like morphology, growing as flat colonies with large nucleo-cytoplasmic ratios, defined borders and prominent nucleoli.

iPSCs express one or more key pluripotency markers known by one of ordinary skill in the art, including but not limited to Alkaline Phosphatase, SSEA3, SSEA4, Sox2, DPPA-2, Oct3/4, Nanog, TRA160, TRA181, TDGF 1, Dnmt3b, FoxD3, GDF3, Cyp26a1, TERT and zfp42.

Somatic cells, with a combination of three, four, five, six, or more factors can be de-differentiated/reprogrammed to a state apparently indistinguishable from embryonic stem cells (ESCs); these reprogrammed cells are termed “induced pluripotent stem cells” (iPSCs, iPCs, iPSCs) and can be produced from a variety of tissues.

The term “stem cell” refers to an unspecialized cell that is capable of replicating or self-renewing itself and developing into specialized cells of a variety of cell types. The product of a stem cell undergoing division is at least one additional cell that has the same capabilities as the original cell. Induced pluripotent stem cells are created by exogenously overexpressing the pluripotency markers (OCT4, SOX2, c-MYC, NANOG and KLF4) using a viral or non-viral vector, thereby inducing pluripotency to the transfected cell line. Pluripotent stem cells are considered to be undifferentiated when they have not committed to a specific lineage. ESCs are considered to be undifferentiated when they have not committed to a specific differentiation lineage. Such cells display morphological characteristics that distinguish them from differentiated cells of embryo or adult origin. Undifferentiated ESCs are easily recognized by those skilled in the art, and typically appear in the two dimensions of a microscopic view in colonies of cells with high nuclear/cytoplasmic ratios and prominent nucleoli. Undifferentiated ESCs express genes that may be used as markers to detect the presence of undifferentiated cells, and whose polypeptide products may be used as markers for negative selection.

Reprogramming: Reprogramming cells using MIP, or any vector that generates similar cancer vaccine properties as to be expected to be the result of the MIP plasmid. Somatic cells of interest for reprogramming include, but are not limited to, fibroblasts, blood cells, urine cells, etc.

The types of cancer that can be treated using the subject methods of the present invention include but are not limited to adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease, e.g., to delay or minimize the growth and spread of cancer. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

Chemotherapy may include Abitrexate (Methotrexate Injection), Abraxane (Paclitaxel Injection), Adcetris (Brentuximab Vedotin Injection), Adriamycin (Doxorubicin), Adrucil Injection (5-FU (fluorouracil)), Afinitor (Everolimus), Afinitor Disperz (Everolimus), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Bexxar (Tositumomab), BiCNU (Carmustine), Blenoxane (Bleomycin), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Dacogen (Decitabine), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Eligard (Leuprolide), Ellence (Ellence (epirubicin)), Eloxatin (Eloxatin (oxaliplatin)), Elspar (Asparaginase), Emcyt (Estramustine), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leukeran (Chlorambucil), Leukine (Sargramostim), Leustatin (Cladribine), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinoIAQ (Cisplatin), PlatinoIAQ (Cisplatin Injection), Pomalyst (Pomalidomide), Prednisone Intensol (Prednisone), Proleukin (Aldesleukin), Purinethol (Mercaptopurine), Reclast (Zoledronic acid), Revlimid (Lenalidomide), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Trelstar (Triptorelin Injection), Trexall (Methotrexate), Trisenox (Arsenic trioxide), Tykerb (lapatinib), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (Ibritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MEDI4736, MPDL3280A and MSB0010718C targeting PD-L1 and those targeting CTLA-4 such as ipilimumab.

Antibiotics, e.g. antibiotics with the classes of aminoglycosides; carbapenems; and the like; penicillins, e.g. penicillin G, penicillin V, methicillin, oxacillin, carbenicillin, nafcillin, ampicillin, etc. penicillins in combination with β-lactamase inhibitors, cephalosporins, e.g. cefaclor, cefazolin, cefuroxime, moxalactam, etc: ; tetracyclines; cephalosporins; quinolones; lincomycins; macrolides; sulfonamides; glycopeptides including the anti-infective antibiotics vancomycin, teicoplanin, telavancin, ramoplanin and decaplanin. Derivatives of vancomycin include, for example, oritavancin and dalbavancin (both lipoglycopeptides). Telavancin is a semi-synthetic lipoglycopeptide derivative of vancomycin (approved by FDA in 2009). Other vancomycin analogs are disclosed, for example, in WO 2015022335 A1 and Chen et al. (2003) PNAS 100 (10): 5658-5663, each herein specifically incorporated by reference. Non-limiting examples of antibiotics include vancomycin, linezolid, azithromycin, daptomycin, colistin, eperezolid, fusidic acid, rifampicin, tetracyclin, fidaxomicin, clindamycin, lincomycin, rifalazil, and clarithromycin.

Radiotherapy means the use of radiation, usually X-rays, to treat illness. X-rays were discovered in 1895 and since then radiation has been used in medicine for diagnosis and investigation (X-rays) and treatment (radiotherapy). Radiotherapy may be from outside the body as external radiotherapy, using X-rays, cobalt irradiation, electrons, and more rarely other particles such as protons. It may also be from within the body as internal radiotherapy, which uses radioactive metals or liquids (isotopes) to treat cancer.

As used herein, endpoints for treatment will be given a meaning as known in the art and as used by the Food and Drug Administration.

Overall survival is defined as the time from randomization until death from any cause, and is measured in the intent-to-treat population. Survival is considered the most reliable cancer endpoint, and when studies can be conducted to adequately assess survival, it is usually the preferred endpoint. This endpoint is precise and easy to measure, documented by the date of death. Bias is not a factor in endpoint measurement. Survival improvement should be analyzed as a risk-benefit analysis to assess clinical benefit. Overall survival can be evaluated in randomized controlled studies. Demonstration of a statistically significant improvement in overall survival can be considered to be clinically significant if the toxicity profile is acceptable, and has often supported new drug approval. A benefit of the methods of the invention can include increased overall survival of patients.

Endpoints that are based on tumor assessments include DFS, ORR, TTP, PFS, and time-to-treatment failure (TTF). The collection and analysis of data on these time-dependent endpoints are based on indirect assessments, calculations, and estimates (e.g., tumor measurements). Disease-Free Survival (DFS) is defined as the time from randomization until recurrence of tumor or death from any cause. The most frequent use of this endpoint is in the adjuvant setting after definitive surgery or radiotherapy. DFS also can be an important endpoint when a large percentage of patients achieve complete responses with chemotherapy.

Objective Response Rate. ORR is defined as the proportion of patients with tumor size reduction of a predefined amount and for a minimum time period. Response duration usually is measured from the time of initial response until documented tumor progression. Generally, the FDA has defined ORR as the sum of partial responses plus complete responses. When defined in this manner, ORR is a direct measure of drug antitumor activity, which can be evaluated in a single-arm study.

Time to Progression and Progression-Free Survival. TTP and PFS have served as primary endpoints for drug approval. TTP is defined as the time from randomization until objective tumor progression; TTP does not include deaths. PFS is defined as the time from randomization until objective tumor progression or death. The precise definition of tumor progression is important and should be carefully detailed in the protocol.

### Methods of Vaccination

Pluripotent stem cells as a source for the cancer vaccine may be obtained from any mammalian species, including, for example, human, primate, equine, bovine, porcine, etc. but particularly human cells.

Cells are reprogrammed to pluripotency. The reprogrammed cells are engineered to knock-out expression of Class I MHC proteins. In an embodiment, gene editing, e.g. CRISPR-Cas9 editing, is utilized to eliminate expression of β2 microglobulin, e.g. by deletion of all or a functional portion of the β2 microglobulin genes in the cell. The cells then lack detectable β2 microglobulin, which is required for cell surface expression of MHC class I proteins, e.g. HLA-A, HLA-B, HLA-C, etc. The hypoallogenic iPSCs are also engineered to knock-out expression of Class II MHC proteins. In an embodiment, gene editing, e.g. CRISPR-Cas9 editing, is utilized to eliminate expression of MHC II transactivator (CIITA) gene, e.g. by deletion of all or a functional portion of the CIITA genes in the cell. The cells then lack detectable CIITA, which regulates expression of Class II MHC proteins, e.g. HLA-DP, HLA-DQ and HLA-DR, etc.

The hypoallogenic iPSCsimmunogenic iPSC are then engineered to over-express CD47. In an embodiment, an expression vector comprising a CD47 coding sequence operably joined to a promoter functional in the iPSC is introduced into the cells. In some embodiments the expression vector is a viral vector, e.g. a lentiviral vector. The cells then over-express CD47.

The hypoallogenic immunogenic iPSCs may be selected prior to vaccine formulation for one or more of (i) decreased Class I MHC expression; (ii) decreased MHC Class II expression; (iii) increased CD47 expression; and (iv) expression of one or more embryonic stem cell markers, e.g. SSEA-1 (CD15/Lewis x), SSEA-3, SSEA-4, DPPA-2, D324 (E-Cadherin), CD90 (Thy-1), CD117 (c-KIT, SCFR), CD326, CD9 (MRP1, TM4SF DRAP-27, p24), CD29 (β1 integrin), CD24 (HAS), CD59 (Protectin), CD133, CD31 (PECAM-1), CD49f (Integrin α6/CD29), TRA-1-60, TRA-1-81, Frizzled5, Stem cell factor (SCF or c-Kit ligand), and Cripto (TDGF-1). In some embodiments the cells are selected for positive expression of an SSEA antigen, e.g. one or more of SSEA1, SSEA-3, SSEA-4.

The engineered hypoallogenic stem cells are grown using standard methods known in the art, such as in feeder cell free conditions until a stable stem cell population is formed. This population should include a >90% pure pluripotent stem cell percentage as assessed by pluripotent stem cell sorting using magnetic antibody sorting (MACS) or fluorescent antibody sorting (FACS).

The hypoallogenic iPSCs may be selected prior to vaccine formulation for one or more of (i) decreased Class I MHC expression; (ii) decreased MHC Class II expression; (iii) increased CD47 expression; and (iv) expression of one or more embryonic stem cell markers, e.g. SSEA-1 (CD15/Lewis x), SSEA-3, SSEA-4, DPPA-2, D324 (E-Cadherin), CD90 (Thy-1), CD117 (c-KIT, SCFR), CD326, CD9 (MRP1, TM4SF DRAP-27, p24), CD29 (β1 integrin), CD24 (HAS), CD59 (Protectin), CD133, CD31 (PECAM-1), CD49f (Integrin α6/CD29), TRA-1-60, TRA-1-81, Frizzled5, Stem cell factor (SCF or c-Kit ligand), and Cripto (TDGF-1). In some embodiments the cells are selected for positive expression of an SSEA antigen, e.g. one or more of SSEA1, SSEA-3, SSEA-4.

The cell dose (range from 1×10⁶ to 1×10⁹) used for the cancer vaccine may need to be adjusted to the mammal that the vaccine is used for. In small rodents, effectiveness of the vaccine was set at 2×10⁶ pluripotent stem cells per dose.

Pluripotent stem cells are usually irradiated prior to vaccination to prevent teratoma formation at the injection site. This dose should be adjusted according to pluripotent stem cell sensitivity or resistance to arresting cell cycle, for example from 1000-10000 rads.

The site of vaccination may be in the subcutaneous space to allow for proper antigen presentation to the immune system. The location of where the vaccine should be placed may change based on the subjects' morphology, but should be performed at different injection sites to avoid local immune suppressive responses. In one embodiment, the method may be performed for a total of four weekly rounds of vaccination over the course of four weeks. In another embodiment, the method may be performed daily, several times a week, or every two weeks, and the duration could be two, three, four, five, six, seven, or eight weeks. The number of vaccinations depends on the subject's immune response towards the vaccine and priming conditions, and therefore, may be adjusted accordingly during treatment.

Small molecular agents or biologic compounds can be used in conjunction with the C+I vaccine to increase the cytotoxic potential of the C+I primed immune cells towards cancer cells. Such molecular agents or biologic compounds may include, for example, diprovocim, a PD-1 or PDL-1 inhibitor, etc.

In one embodiment, a therapeutic dose of the adjuvant is provided, and will depend on the adjuvant being used for the cancer vaccine. Depending on the mammal and type of cancer being treated, a factor of 10 dilution or concentration of the adjuvant may be used, such as a concentration of 0.05 μM, 0.03 μM, 0.01 μM; or 10 μM, 30 μM, or about 50 μM. It will be understood by one of skill in the art that such guidelines will be adjusted for the molecular weight of the active agent, and also based on the effectiveness of the adjuvant for the specific treatment. The dosage may also be varied for type of mammal receiving the vaccine.

For parenteral administration, compositions of the invention can be administered as injectable dosages of a solution or suspension of the substance in a physiologically acceptable diluent with a pharmaceutical carrier that can be a sterile liquid such as water, oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

Typically, compositions are prepared as injectables, either as liquid solutions or suspensions; solid forms suitable for solution in, or suspension in, liquid vehicles prior to injection can also be prepared. The pharmaceutical compositions are generally formulated as sterile, substantially isotonic and in full compliance with all Good Manufacturing Practice (GMP) regulations of the U.S. Food and Drug Administration. Preferably, a therapeutically effective dose will provide therapeutic benefit without causing substantial toxicity.

In another embodiment, there is provided a thermally stable vaccine composition comprising an effective amount of hypoallogenic iPSCs as disclosed herein, and optionally, an adjuvant or an immunological agent to boost the immune response towards the vaccine. In one variation, the thermally stable vaccine or heat stable vaccine allows for storage that does not require cold chain storage, allowing the facile introduction of the vaccines in areas with no or limited cold chain storage capacity. In one variation, the vaccine further comprises an effective amount (such as an approximate range of 0.01% to 1% wt/wt, 0.05% to 0.5% wt/wt, 0.05% to 1% wt/wt, or 0.01% to 0.5% wt/wt) of a glycol, such as propylene glycol, polyethylene glycol 300 and glycerin, or mixtures thereof. In another variation, the vaccine is stable for up to 6 months, up to 12 months, up to 24 months or up to 36 months at about 35 °C, either as a standard liquid formulation or as a spray dried formulation.

One aspect of the drying method for the vaccine includes a spray drying method. The spray-drying method may include, for example, a method for spraying from a high-pressure nozzle, or by using a centrifugal force, such as an atomizer as known in the art. The gas or air that may be used for the spray drying includes heated air or hot air at a temperature sufficient to dry the vaccine powder having the desired moisture content. In one aspect, the gas is an inert gas such as nitrogen or nitrogen-enriched air.

In one aspect, the hot gas temperature may be at about 30 °C to 50 °C, 30 °C to 60 °C, 30 °C to 70 °C, or about 30 °C to 100 °C. The high pressure that may be used for the spray during process used in a high pressure nozzle may include about 10 to 1,000 psi, 100 to 800 psi or 200 to 500 psi. The spray drying may be carried out under conditions such that the residual water or residual moisture content of the dry vaccine may be controlled to about 1% to about 6%, 1% to 5%, 2% to 6%, 3% to 6% or about 3% to 5%.

In one aspect, the emulsions may then be sprayed dried in conventional spray drying equipment from commercial suppliers, such as Buchi, Niro, Yamato Chemical Co., Okawara Kakoki Co., and similar commercially available spray drier. Spray drying processes, such as rotary atomization, pressure atomization and two-fluid atomization may also be used. Examples of the devices used in these processes include Parubisu Mini-Spray GA-32 and Parubisu Spray Drier DL-41 (Yamato Chemical Co.) or Spray Drier CL-8, Spray Drier L-8, Spray Drier FL-12, Spray Drier FL-16 or Spray Drier FL-20, (Okawara Kakoki Co.), may be used for the spray drying method using rotary-disk atomizer. The nozzle of the atomizer that produces the powder of the present application may include, for example, nozzle types 1A, 1, 2A, 2, 3 (Yamato Chemical Co.) or similar commercially available nozzles, may be used for the above-mentioned spray drier. In addition, disks type MC-50, MC-65 or MC-85 (Okawara Kakoki Co.) may be used as rotary disks of the spray-drier atomizer.

In another aspect, the vaccine powder obtained from the drying process may comprise 1% by weight, 5% by weight, 7% by weight, 10% by weight, 20% by weight, 30% by weight, 40% by weight, 50% by weight or more of particles having an average particle size in the range from about 5 to 1,000 microns, from about 10 to 500 microns, from 10 to 350 microns, from 20 to 250 microns, from 40 to 200 microns, or about 50 to 150 microns. In one aspect, the powder obtained from the drying process comprises of about 1% to 10% by weight of particles with an average particle size of 50 to 150 microns.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. It is also understood that the terminology used herein is for the purposes of describing particular embodiments.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or only and is not intended to limit the scope of the present invention which will be limited only by the appended claims.

Those skilled in the art will recognize or be able to ascertain using no more than routine experimentation, many equivalents to the specific embodiments of the invention described herein. Such equivalents are intended to be encompassed by the appended claims.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The B2m gene in C57BL/6 iPSC cells (miPSCs) was knocked out using B2m Mouse Gene Knockout Kit (Synthego). The resultant miPSCs colonies were then sequenced by PCR and analyzed through Synthego ICE software to select for the single colonies that have knockout score around 100. The B2m knockout single colony was then expanded and tested for pluripotency before proceeding to Ciita gene Knockout, and a similar procedure (Synthego Ciita Mouse Gene Knockout Kit for Ciita gene depletion and Synthego ICE software for PCR sequencing analysis) was performed to generate the B2m^{-/-}Ciita^{-/-} miPSCs. After verifying the pluripotency status of the B2m^{-/-}Ciita^{-/-} miPSCs, the mouse Cd47 lentiviral particles were utilized, which have the Cd47 DNA cloned into a lentiviral vector with puromycin resistance (Origene), for transducing B2m^{-/-}Ciita^{-/-} miPSCs with the presence of puromycin. The B2m^{-/-}Ciita^{-/-} Cd47 tg miPSCs were selected and expanded as the hypoallogeneic iPSCs.

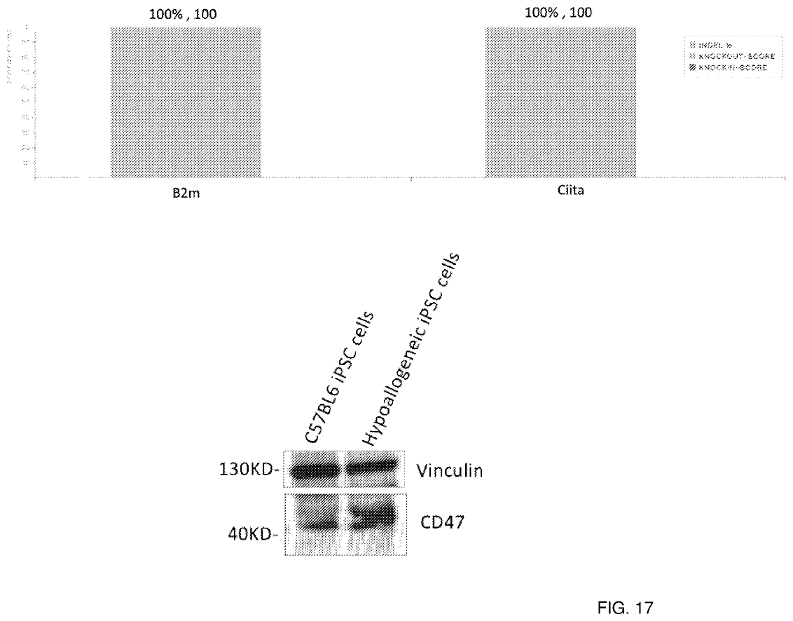

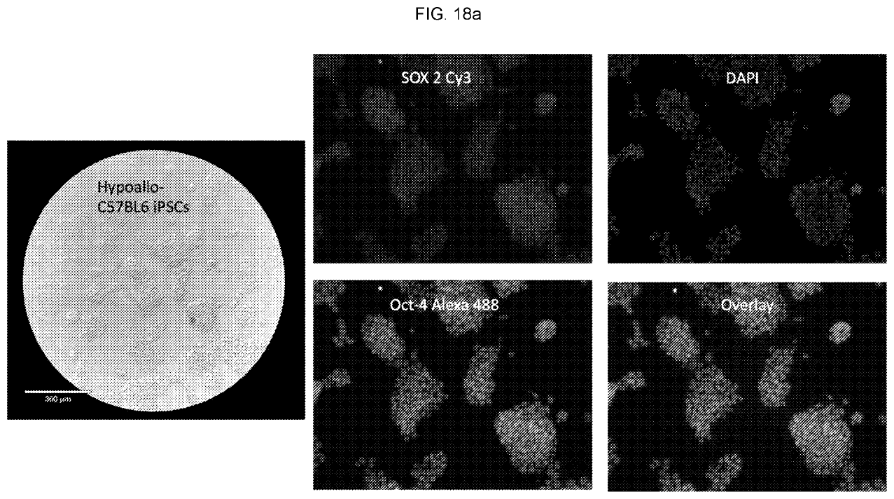

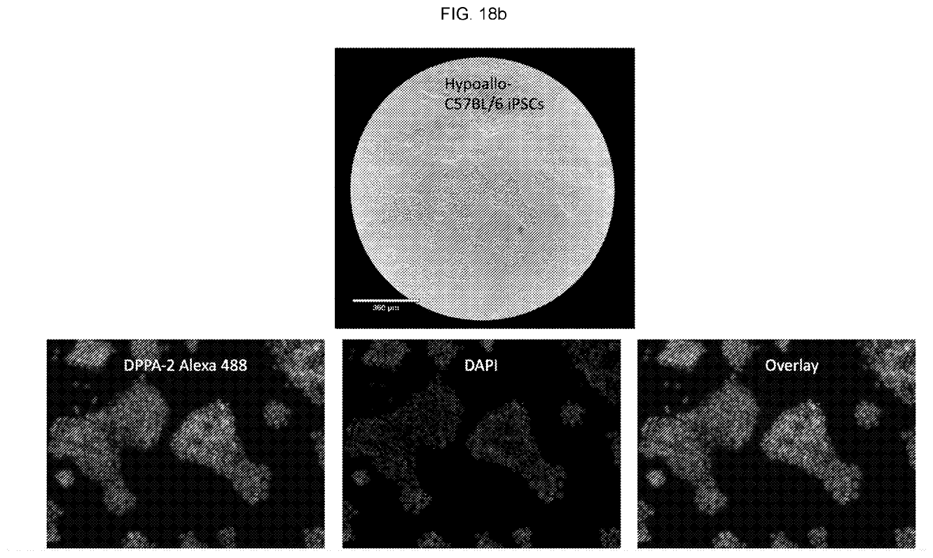

DNA were extracted from control C57BL6 iPSC cells and hypoallo-immunogenic C57BL6 iPSC cells for PCR-amplify of the genomic regions that were targeted by b2m and ciita sgRNAs. The resulting PCR products were then submitted for Sanger sequencing. Synthego ICE software analysis of the PCR sequencing data files has shown that the overall b2m and ciita gene knockout efficiency in hypoallo-immunogeneic C57BL6 iPSC cellscells are 100. Western blot analysis of total protein lysates from control C57BL6 iPSCs and hypoallo-immunogenic C57BL6 iPSCs confirmed that CD47 protein has been overexpressed in hypoallo-immunogenic C57BL6 iPSC line (vinculin was used as western blot loading control). (FIG. 17) The pluripotency status of the generated hypoallo-immunogenic C57BL6 iPSC line were also confirmed by immunofluorescence staining for pluripotent transcription factors Oct4, Sox2, and DPPA2. As shown in FIG. 18, these newly recovered lines maintained high expression of pluripotency markers.

CpG+iPSC vaccine preparation and immunization. For each mouse, 2×10⁶ SSEA-1-sorted syngeneic murine iPSCs (autologous, hypoallogeneic or allogenic) were irradiated at 6,000 rads prior to injection. Cells were suspended in 100 μL of 5 μM CpG (Invivogen, San Diego, USA), dissolved in PBS, and loaded into ¼ cc insulin syringes (Terumo). Mice were placed in an induction chamber and anesthetized with 2% isoflurane (Isothesia, Butler Schein) in 100% oxygen with a delivery rate of 2 l/min until the loss of righting reflex, as per APLAC guidelines at Stanford University. Immunization was performed by subcutaneous injection of the vaccine in the flanks of the mice, with the injection site changing every week. Mice were monitored weekly for early signs of auto-reactivity to the vaccine by weight measurements and gross examination of overall appearance.

Cancer cell lines and implantation. The colorectal cancer line CT26 were grown in RPMI, 10% FBS under normal culture conditions. 5×10⁵ cancer cells were resuspended in 100 μL PBS and injected subcutaneously in right flank of the mice. Tumor growth was assessed every three days by caliper measurement, and all mice will be sacrificed simultaneously when any mouse has tumor size reaches 2 cm³.

Biospecimen Collection and Histopathology analysis. At time of sacrifice (day 21 post-tumor implantation), the brain, thymus, salivary gland, heart, lung, liver, pancreas, GI tract, ovary, uterus, and kidneys, skin, muscle were isolated from mice and processed for histopathology. Briefly, the organs were fixed overnight in 4% paraformaldehyde and transferred to 70% ethanol for 24 hr. Fixed samples were embedded in paraffin and 5 μm sections were cut and stained with hematoxylin and eosin (H&E) for histological analysis by a pathologist.

T cell Purification. At time of sacrifice (day 21 post-tumor implantation), the spleen was isolated from mice and put in 10% FBS containing RPMI medium on ice. The flat end of the plunger was then applied to mince spleen to release cells, which were filtered through a 70 μm strainer, spun down, and resuspended in ACK lysis buffer to remove any red blood cells. After lysis, the cells suspension was washed once with 10% FBS containing RPMI medium and then froze down at −80 degree until use.

In vitro T cell stimulation. 0.5E6 cells per well of isolated mouse splenocytes were seeded onto each well of 48 well tissue culture plate with either the absence or presence of 70 ug/mL of CT26 tumor lysate dissolved in 10% FBS containing RPMI medium. The experiment was performed in triplicate for each splenocyte sample, and the cell number was counted using Flow Cytometer after 48 hours of incubation at 37 degrees.

Elispot Assay. Isolated splenocytes were co-cultured with CT26 tumor lysate (35 μg) for the duration of 40 hr, after which the secretion of granzyme-β and IFN-γ was measured by Enzyme-Linked ImmunoSpot (ELISPOT) according to the manufacturer's instructions (cat #EL485, EL1865, R&D Systems). BioTek Gen 5 and Image J were used for analyzing the number of IFN-γ and granzyme B positive spots.

IgG binding assay. 0.5E6 of CT26 cells were washed multiple times with PBS and resuspended in 200 μL FACS buffer with the addition of 2 μL of serum from mice after 4 weeks of vaccination and incubated for 30 min on 4 °C. Following this, cells were washed multiple times and incubated with an anti-IgG Alexa Fluor® 647 secondary antibody (ThermoFisher Scientific) for another 30 min on 4 °C. The cells were then washed twice with FACS buffer and then resuspend in 400 uL of FACS buffer for Flow Cytometer analysis.

Total IgG level measurement. Collected mouse serum were diluted and tested using Easy-Titer Mouse igG Assay kit (ThermoFisher) to measure the total IgG concentration in serum.

Mouse weight measurement. The changes of weight of mice immunized with vaccine were closely monitored up to twice per week before, during and after the administration of vaccines.

Anti-nuclear antibody (ANA) enzyme-linked immunosorbent assay (ELISA): The vaccinated mouse serum samples will be diluted at 1:200 with sample dilution. The concentrations of anti-nuclear antibodies (IgG) were determined using an ELISA kit, according to the manufacturer's instructions (Biomatik).

Establishment of Orthotopic Triple Negative Breast Cancer Tumor. 4T1-Luc-GFP cells were harvested from cell culture and resuspended in PBS at a density of 2E5 cells/ml. Then 50 uL of tumor cells were injected into the mammary fat pad under #3 mammary gland of BALB/c mouse.

Surgical Removal of Primary Breast Tumors. Breast tumor growth were measured every three days after initial tumor inoculation. When breast tumor size reached 800 to 1000 mm³, mice were anesthetized under constant flow of 2% isoflurane and oxygen. The tumor area was sterilized with 70% ethanol. The mouse skin around the tumor was excised to expose the tumor which was removed with a pair of sterile scissors.

Live mouse Bioluminescence Imaging. Mice were imaged every 3 days after primary tumor removal. 200 μl luciferin (15 mg/ml in PBS) were injected intraperitoneally (i.p.) to mice prior to imaging. After 5 minutes, anesthetize mice in an induction chamber with 2% isoflurane/O2 flow were transferred to imaging chamber with continuous 2% isoflurane and O2 at a flow rate of 2 L/min. Bioluminescence imaging were acquired with exposure time at 60 seconds (or 30 seconds if image saturation occurred) and field of view (FOV) at 25, object height at 1.5 cm, and luminescent photographic images were obtained with low power X-ray. The units of measurement were set to “radiance.”

Establishment of B cell lymphoma in mice. A20 cells were harvested from cell culture and resuspended in PBS at a density of 1.5 E7 cells/ml, then 100 uL of tumor cells were injected into the right upper flank of BALB/c mouse.

### Results

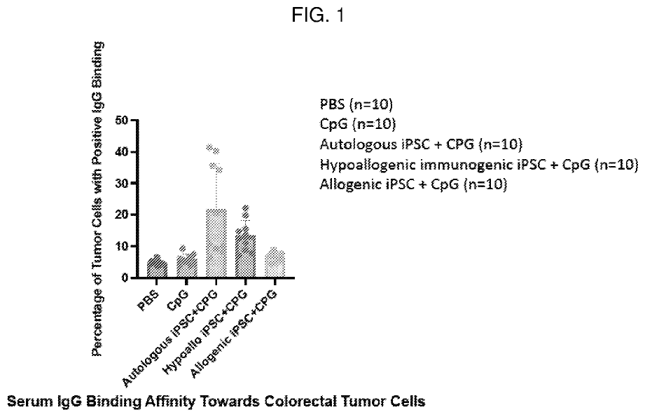

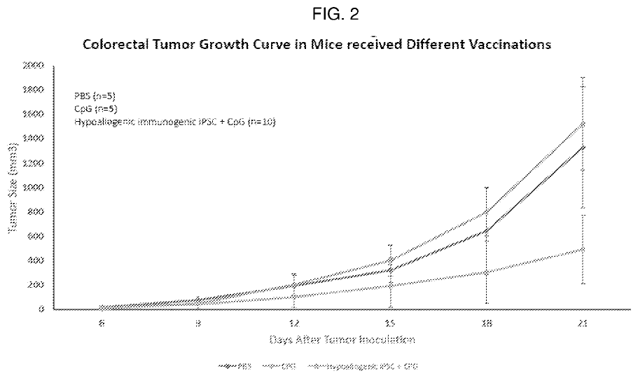

As shown in FIG. 1, hypoallogenic immunogenic ipscs-based vaccine induces anti-colorectal tumor specific IgG Antibody secretion in mice after 4 weeks of immunization before tumor challenge. Mice that received hypoallogenic immunogenic iPSC-based vaccine have slower tumor growth after syngeneic colorectal tumor challenge, shown in FIG. 2.

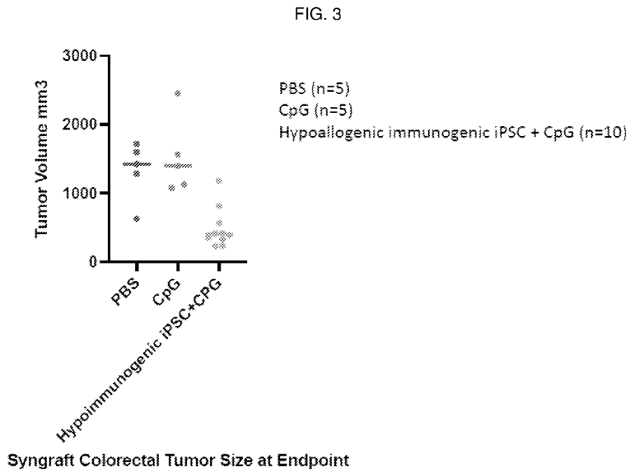

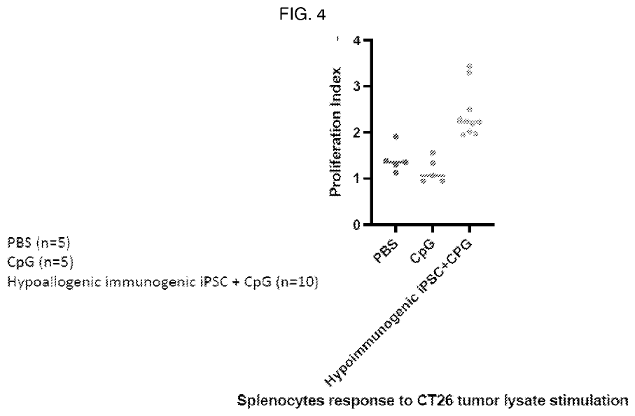

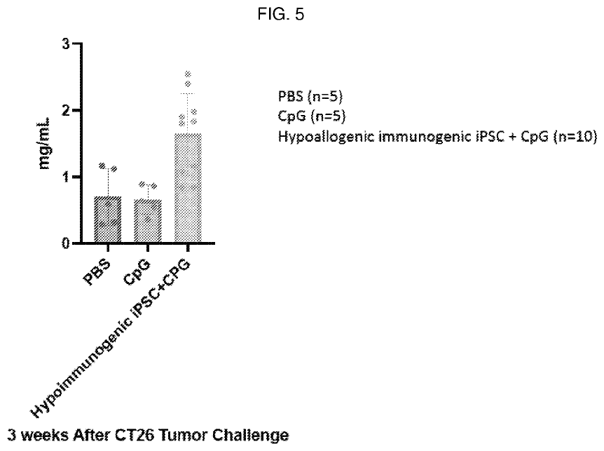

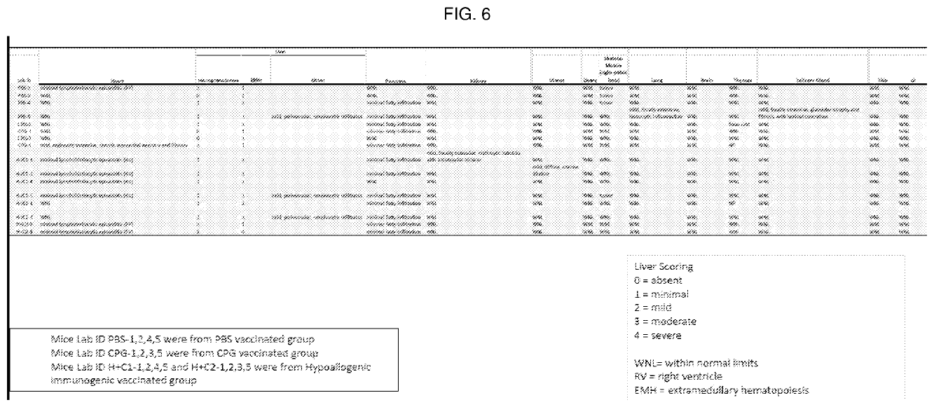

Hypoallogenic immunogenic iPSC-based vaccine inhibits colorectal tumor growth, shown in FIG. 3, and splenocytes from ct26 tumor-bearing mice that pre-vaccinated with hypoallogenic immunogenic iPSC-based vaccine have significant increase in proliferation upon in-vitro colorectal tumor lysate stimulation (FIG. 4); and the vaccine promoted total IgG secretion in tumor bearing mice (FIG. 5). No vaccine-related toxicities were identified in necropsy analysis of mice immunized with hypoallogenic immunogenic iPSC-based vaccine (FIG. 6).

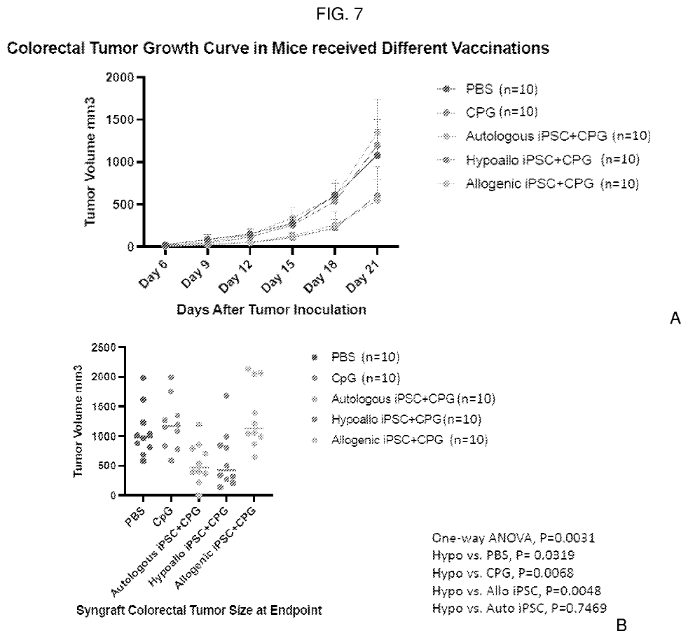

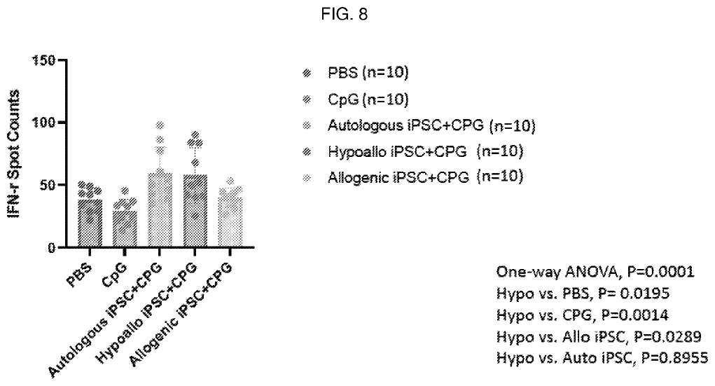

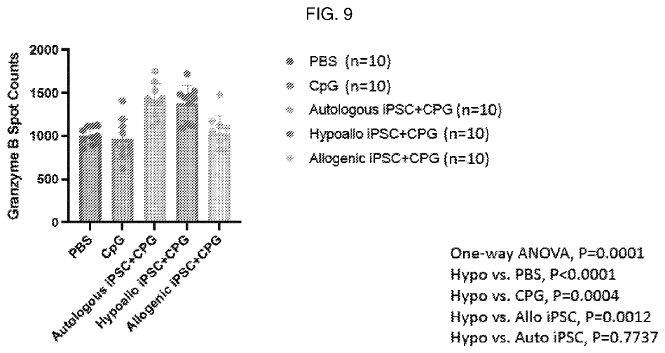

Hypoallogenic immunogenic iPSC-based vaccine shows a similar anti-cancer efficacy as autologous iPSC-based vaccine (FIG. 7); and similar to the autologous-iPSC vaccinated Group, hypoallogeneic iPSC-vaccinated group produced higher numbers of IFN-γ spots than the other vaccination groups under in-vitro colorectal tumor lysate stimulation (FIG. 8); and produces higher numbers of granzyme b spots than other vaccination groups under in-vitro colorectal tumor lysate stimulation (FIG. 9).

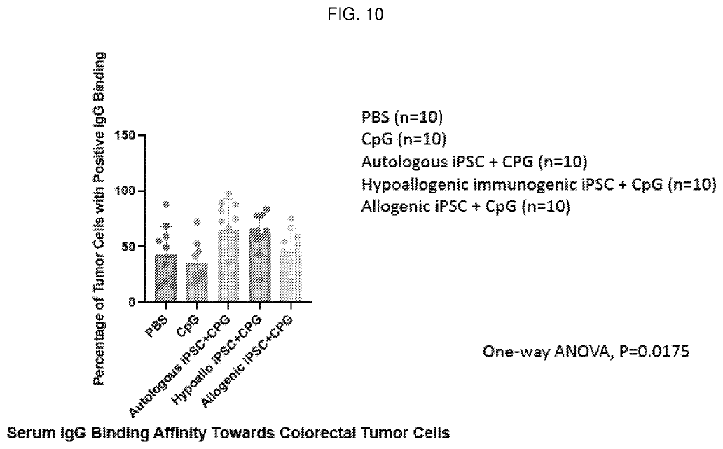

Shown in FIG. 10, hypoallogenic immunogenic iPSCs-based vaccine induces anti-colorectal tumor specific IgG antibody secretion in mice 3 weeks post-tumor challenge.

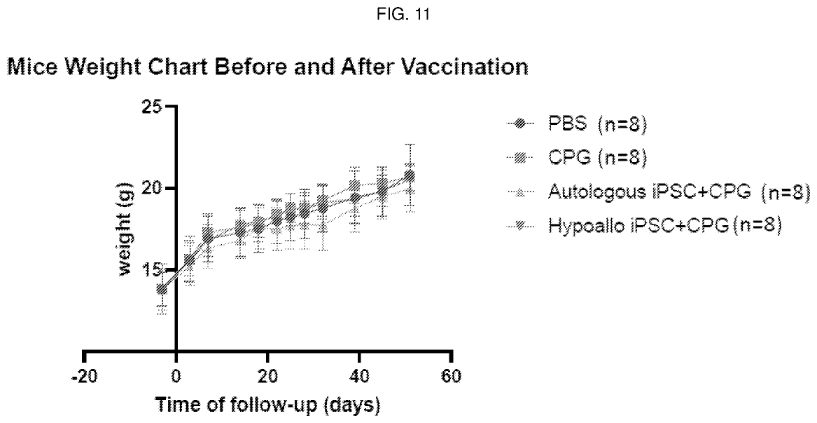

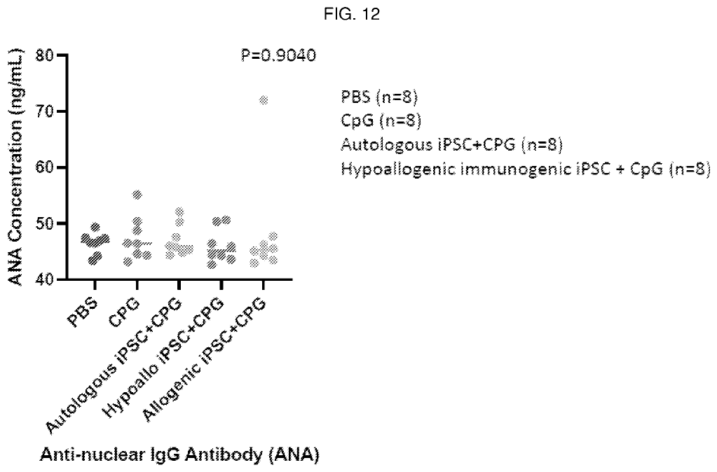

With respect to side effects, it was found that the hypoallogenic iPSCs-based vaccine dose not induce significant change in mice weight either during or after administration of vaccination and does not significantly increase serum concentration of anti-nuclear antibody (ANA), which indicates that no autoimmune response are triggered by the vaccine (FIGS. 11 and 12).

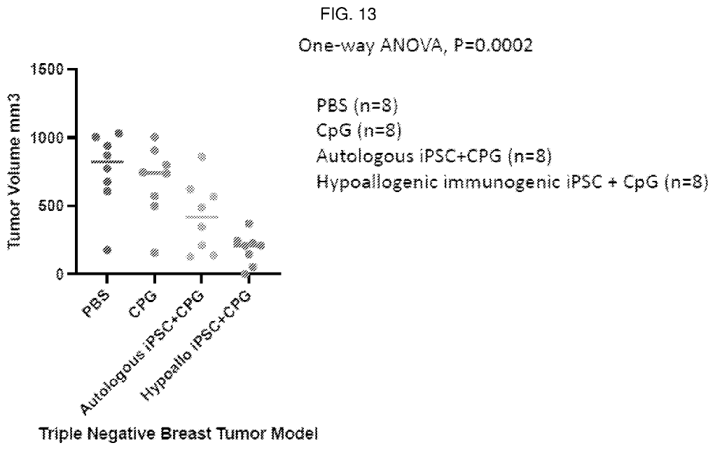

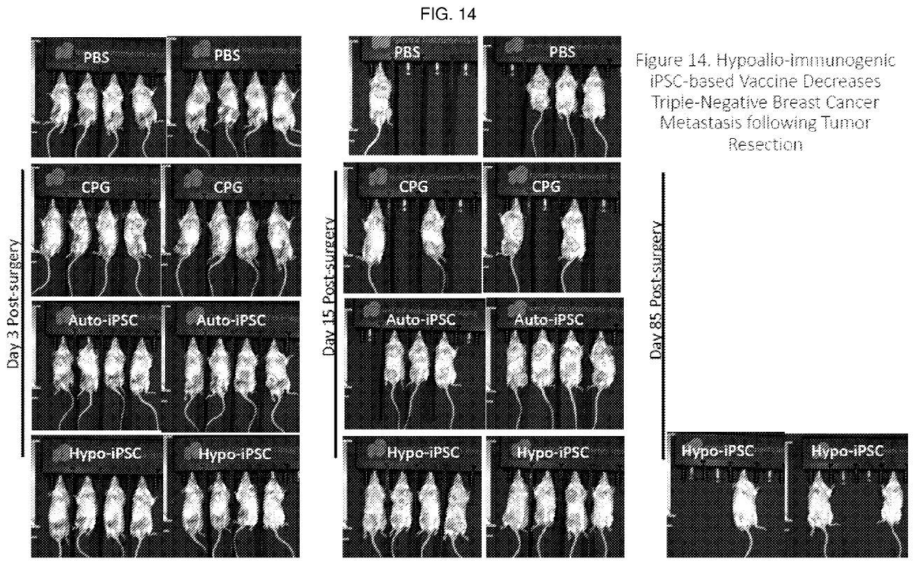

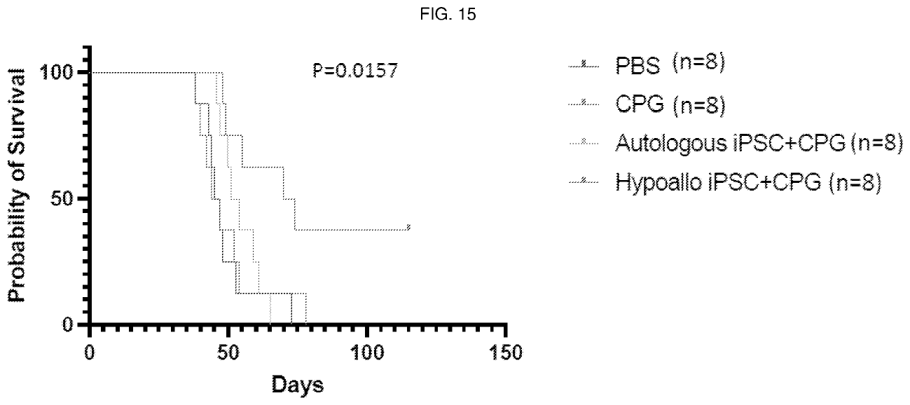

30 days after triple negative breast tumor challenge, hypoallogenic immunogenic iPSCs vaccinated mice have the slowest tumor growth among all groups (FIG. 13). After primary 4T1 breast tumor removal, mice were imaged every 3 days to track for secondary organ metastases. Bioluminescence images taken at day 3, day 15 and day 85 post-primary tumor resection were shown in the figure. At the study endpoint (day 85 post-surgical removal of primary tumor), mice in other vaccination groups have all developed metastases, except for the three mice in hypoallo-immunogenic iPSCs vaccination group (FIG. 14). By the end of the study (day 115 post-breast tumor challenge or day 85 post-surgical removal of primary tumor), three out of eight mice in hypoallo-immunogenic iPSC vaccinated group are still alive with no signs of cancer relapse (FIG. 15).

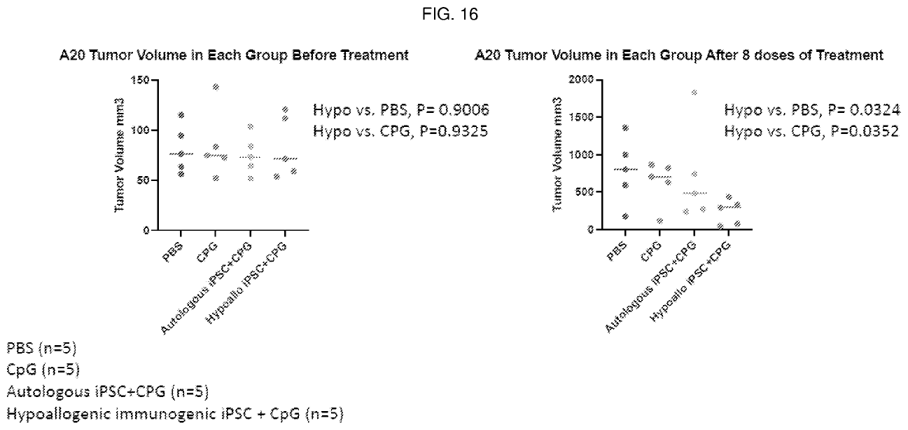

Mice were inoculated with A20 tumors. After the tumor volume reached 50 to 100 mm3, mice were randomly distributed to different treatment groups (PBS, CPG, autologous iPSC-based vaccine, hypoallo-immunogenic iPSC-based vaccine) to receive subcutaneously injection three times per week. Before application of treatment, there was no difference in tumor size among all groups. By the end of the study, mice in hypoallo-immunogenic iPSC treatment group have the smallest tumor size comparing to other groups (FIG. 16).

DNA were extracted from control C57BL6 iPSC cells and hypoallo-immunogenic C57BL6 iPSC cells for PCR-amplify of the genomic regions that were targeted by b2m and ciita sgRNAs. The resulted PCR products were then submitted for Sanger sequencing. Synthego ICE software analysis of the PCR sequencing data files has shown that the overall b2m and ciita gene knockout efficiency in hypoallo-immunogeneic C57BL6 iPSC cells are 100. Western blot analysis of total protein lysates from control C57BL6 iPSCs and hypoallo-immunogenic C57BL6 iPSCs confirmed that CD47 protein has been overexpressed in hypoallo-immunogenic C57BL6 iPSC line. Vinculin was used as western blot loading control (FIG. 17).

Hypoallo-immunogenic C57BL6 iPSC cells maintained high expression levels of pluripotency markers: Oct4, Sox2, and DPPA2 (FIG. 18).

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method for the treatment of cancer in a patient, the method comprises a vaccination of the patient with a vaccine, wherein the vaccine comprises an effective amount of allogeneic hypoallogenic mammalian pluripotent stem cells, wherein the vaccination comprising the step of administering mammalian pluripotent stem cells to the patient in need thereof.

2. The method of claim 1 wherein the pluripotent stem cells are induced pluripotent stem cells (iPSCs) wherein one of more of MHCI protein expression is inhibited; MHCII protein expression is inhibited; and CD47 is overexpressed.

3. The method of claim 1, wherein the mammalian pluripotent stem cells comprise pluripotent stem cells comprising
one or more alterations that inactivate both alleles of an endogenous B2M gene;
one or more alterations that inactivate both alleles of an endogenous CIITA gene; and
one or more alterations causing an increased expression of a CD47 gene.

4. The method of claim 1, wherein the pluripotent stem cells are generated using a mini-intronic plasmid or vectors based on a modified, non-transmissble form of Sendai virus (SeV) to deliver and express four reprogramming factors comprising Oct4, c-Myc, KLF-4 and Sox2.

5. The method of claim 1, further comprising shRNA p53 in the mini-intronic plasmid.

6. The method of claim 1, wherein the stem cells are obtained by reprogramming somatic cells selected from the group consisting of fibroblast, keratinocytes, peripheral blood cells and renal epithelial cells.

7. The method of claim 1, wherein the stem cells are allogenic.

8. The method of claim 1, wherein the vaccine is administered according to at least one of the following methods: a) as a standalone vaccination; b) as an adjuvant therapy before tumor resection; c) as an adjuvant therapy after tumor resection, d) in a metastatic setting; e) as a preventative setting in the absence of tumor or cancer; and f) in combination with chemotherapy, immunotherapy, targeted therapies, using biologic agents, using small molecule agents, with nanoparticles comprising the biologic or small molecule agents, or a combination thereof.

9. The method of claim 1, wherein the cancer is selected from the group consisting of breast cancer, melanoma and mesothelioma, leukemia, multiple myeloma, Hodgkin's lymphoma, non-Hodgkin's lymphoma, lymphoma, myeloproliferative disorders, squamous cell cancer, adenocarcinoma, sarcoma, neuroendocrine carcinoma, bladder cancer, skin cancer, brain and spinal cord cancers, head and neck cancer, thyroid, bone cancer, breast cancer, cervical cancer, endometrial cancer, gastrointestinal cancers, (hypo) laryngeal cancer, esophageal cancer, liver cancer, lung cancer, pancreatic cancer, prostate cancer, eye cancer, renal cell cancer, kidney, hepatic, ovarian cancer, gastric cancer, testicular cancer, thyroid and thymus cancer.

10. The method of claim 1, wherein the cancer is colorectal cancer.

11. The method of claim 1 wherein the vaccine composition comprises an adjuvant that is an immunological agent to boost the immune response towards the vaccine.

12. The method of claim 11 wherein the adjuvant is selected from the group consisting of CpG, QS21, poly(di(carboxylatophenoxy)phosphazene; derivatives of lipopolysaccharides such as monophosphoryl lipid A, muramyl dipeptide (MDP; Ribi), threonyl-muramyl dipeptide (t-MDP; Ribi); OM-174; cholera toxin (CT), and Leishmania elongation factor.

13. The method of claim 11, wherein an adjuvant is selected from oil-in-water emulsion-based adjuvants (MF59, AS03), adjuvants containing the TLR4 agonist 3-O-desacyl-4′-monophosphoryl lipid A (MPL) (AS01, AS04), and CpG 1018, a TLR9 agonist CpG oligonucleotide.

14. The method of claim 1, wherein the vaccine is irradiated prior to vaccination.

15. The method of claim 1, wherein the iPSCs are selected for expression of an embryonic antigen prior to vaccination.

16. A vaccine composition for use in claim 1.
